(19) United States
(12) Patent Application Publication
(10) Pub. No.: US 2005/0142218 A1
(43) Pub. Date: Jun. 30, 2005

(54) Pharmaceutical composition containing nitrate source and an acidifying agent for treating skin ischaemia

(75) Inventors: Arthur T. Tucker, London (GB); Nigel Benjamin, London (GB)

Correspondence Address: OSHA LIANG L.L.P., 1221 MCKINNEY STREET, SUITE 2800, HOUSTON, TX, 77010, US

(73) Assignee: Queen Mary & Westfield College, London (GB)

(21) Appl. No.: 11/065,813

(22) Filed: Feb. 25, 2005

Related U.S. Application Data
(63) Continuation of application No. 09/949,202, filed on Sep. 7, 2001.
Application No. 09/949,202 is a continuation of application No. PCT/GB00/00853, filed on Mar. 9, 2000.

(51) Int. Cl. A61K 33/00

(52) U.S. Cl. 424/718

(57) ABSTRACT

The use of acidified nitrate as an agent to produce local production of nitrate oxide at the skin surface is described in the treatment of peripheral ischaemia and associated conditions. The dosage form may be in any pharmaceutically acceptable carrier means and comprises an acidifying agent adapted to reduce the pH at the environment. A barrier consisting of a membrane allows diffusions of the nitrate ions while preventing direct contact of the skin and acidifying agent. Amongst the many potential applications for the invention is the management of chronic skin wounds, peripheral ischaemia conditions such as Raynaud's phenomenon. Compositions and methods of use for these applications are described.

### CROSS-REFERENCE TO RELATED APPLICATIONS

This is a continuation of and claims benefit from U.S. application Ser. No. 09/949,202 filed on Sep. 7, 2001, which is a continuation of PCT Application No. PCT/GB00/00853, filed on Mar. 9, 2000, which claims priority from G.B. Application No. 9905425.6, filed on Mar. 9. 1999.

### BACKGROUND

1. Field of the Invention

The present invention relates to a new pharmaceutical use of acidified nitrite contained within a delivery system which allows passage of nitric oxide to the skin as a treatment for ischaemic ulceration, to promote wound healing and associated conditions.

2. Background of the Invention

Nitric oxide [NO] is a potent vasodilator synthesised and released by vascular endothelial cells and plays an important role in regulating vascular local resistance and blood flow. In mammalian cells,-NO is principally produced along with L-citruilline by the enzymatic oxidation of L-arginine. Nitric oxide is also involved in the inhibition of both platelet and leukocyte aggregation and adhesion, the inhibition of cell proliferation, the scavenging of superoxide radicals and the modulation of endothelial layer permeability. Nitric oxide also has been shown to possess anti-microbial properties, reviewed by F. C. Fang (1997) (J. min. Invest. 99 (12) 2818-2825 (1997)).

A potential therapeutic utility of the anti-microbial properties of NO is described in WO 95/122335. A pharmaceutical composition comprising nitrite in an inert carrier cream or ointment and salicylic acid was used to show killing of cultures containing E. coli and C. albicans. This activity was further tested against patients with fungal infection of the feet (“Athlete's Foot” or tidea pedis) and showed that the condition was amenable to treatment with the acidified nitrite composition. However, the composition of nitrite and organic acid caused erythema (redness) of the skin.

In addition to internal cell-mediated production, NO is also continually released externally from the surface of the skin by a mechanism which appears to be independent of NO synthase enzyme. Nitrate excreted in sweat is reduced to nitrite by an unknown mechanism which may involve nitrite reductase enzymes which are expressed by skin commensal bacteria. Alternatively mammalian nitrite reductase enzymes may be present in the skin which could reduce nitrite rapidly to NO on the skin surface.

The production of NO from nitrite is believed to be through the following mechanism:
NO₂⁻+H⁺ [symbol] HNO₂ [1]
2HNO₂ [symbol] N₂O₃+H₂O [2]
N₂O₃ [symbol] NO+NO₂ [3]

Although the amount of NO generated by this physiological mechanism is not sufficient to affect skin blood flow, it is clear that very large amounts of NO can be generated by the topical application of nitrite and acid.

### SUMMARY OF THE INVENTION

It has now been surprisingly found that topical application to the skin of nitrite at concentrations of up to 4% in an inert carrier cream or ointment when mixed with an organic acid such as ascorbic acid (vitamin C) reacts to produce oxides of nitrogen to cause the release of nitric oxides leading to sustained vasodilation of the microcirculatory blood vessels, without significant inflammation. This new use for acidified compositions containing nitrite is a departure from the previously known uses of the composition as an anti-microbial agent. The side-effects of erythema and irritation to the skin from the acid in the composition associated with the treatment of fungal infections of the foot had been considered to suggest that the composition should not be used on broken skin or away from sites of infection needing immediate, short term therapeutic treatment. Additionally, the skin on the foot is significantly thicker and tougher than elsewhere on the mammalian body and so can endure more prolonged erythema than other thinner areas of skin elsewhere. Furthermore there is a widespread and generally accepted medical prejudice against inserting ointments or gels into open wounds or onto broken skin. Such practice is advised against because of the risk of actually causing infection or septicaemia (blood-poisoning).

The ability of the composition to cause vasodilation is also surprising because the NO molecule would not normally be expected to cross the outer layers of the skin into the inner layers of the epidermis to act on the blood vessels and microcapillaries.

According to a first aspect of the invention there is provided the use of a pharmacologically acceptable acidifying agent, a pharmacologically acceptable source of nitrite ions or a nitrite precursor therefore in the preparation of an agent for the treatment of skin ischaemia and associated conditions.

The pharmacologically acceptable acidifying agent is adapted to reduce the pH at the site of application and can include any suitable organic acid such as ascorbic acid (vitamin C), salicylic acid, acetyl salicylic acid, acetic acid or a salt or a derivative thereof in a concentration up to 20% w/w, suitably 0.25 to 10% w/w, preferably 4 to 6% w/w. A particularly preferred concentration is 4% or 5% w/w. The preferred pH range is from pH2 to pH7, preferably pH4. Other acidifying agents include but are not limited to, ammonium or aluminium salts, phenol, benzoic acid. Inorganic acids such as hydrochloric acid may be used if sufficient dilute and/or appropriately buffered. The acidifying agent may be present as a dissolved salt or in a liquid form.

The pharmacologically acceptable source of nitrite ions may be an alkaline metal nitrite or an alkaline earth metal nitrite, For example, LiNO₂, NaNO₂, KNO2, RbNO₂, CsNO₂, FrNO₂, Be(NO)₂, Mg(NO₂)₂, Ca(NO₂)₂, Sr(NO₂)₂, Ba(NO₂)₂, or Ra(NO₂)₂. Alternatively, a nitrite precursor may be used as the source of the nitrite ions in the composition, such as for example a dilute solution of nitrous acid. Other sources of nitrite ions are nitrate ions derived from alkali metal or alkaline earth metal salts capable of enzymic conversion to nitrite. For example, LiNO₃, NaNO₃, KNO₃, RbNO₃, CsNO₃, FrNO₃, Be(NO₃)₂, Mg(NO₃)₂, Ca(NO₃)₂, Sr(NO₃)₂, Ba(NO₃)₂ or Ra(NO₃)₂. The concentration of the nitrate ion source may be up to 20% w/w, suitably 0.25 to 10%, preferably 4 to 6%. A particularly preferred concentration is 4% or 5% w/w. The pharmacologically acceptable sources of nitrite ions or a nitrite precursor, e.g., nitrates, will be generally referred to herein as the “pharmacologically acceptable source of nitrite.”

Suitably, the final nitrite ion concentration present in the composition is up to 20% w/w, generally in the range of from 0.25% to 15% w/w, suitably 2% to 10% w/w, preferably 4 to 6% w/w. A particularly preferred nitrite ion concentration is 4% or 5% w/w.

Ischaemia is defined as an inadequate or impaired blood flow to a part of the body. The present invention seeks to provide the use of a composition in the treatment of skin ischaemia and its associated peripheral skin conditions. For example, disease conditions such as Raynaud's phenomenon and severe primary vasospasm are characterised by poor blood flow to the skin. Damage to the skin of an individual also leads to skin ischaemia as the blood supply is reduced or prevented by the body's own repair or defence mechanisms.

Ischaemic skin conditions which may benefit from the therapeutic use of a composition as defined in accordance with this aspect of the invention, include but are not limited to wounds, including skin ulcers and post-operative trauma, bums. This aspect of the invention therefore also extends to platelet and/or leukocyte aggregation and adhesion, cell proliferation, scavenging of superoxide radicals and endothelial layer permeability. Other dermatological conditions such as acne associated with skin ischaemia can also be treated by these compositions.

In the preparation of an agent according to this aspect of the invention, the acidifying agent and the nitrite ions or source therefore are formulated in a pharmacologically acceptable carrier or diluent which may be an inert cream or ointment. In a particular preferred form of the invention the acidifying agent and the source of nitrite ions or precursor therefore are separately disposed in the said cream or ointment for admixture to release ions at the environment of use.

The pharmaceutical composition may be adapted for administration by any appropriate topical route, including buccal, sublingual or transdermal. Such Compositions may be prepared by any method known in the art of pharmacy, for example by admixing the active ingredient with the carrier(s) or excipient(s) under sterile conditions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):319 (1986).

Pharmaceutical compositions adapted for topical administration may be formulated as ointments, creams, suspensions, lotions, powders, solutions, pastes, gels, sprays, aerosols or oils. For treatment of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the substance of the present invention.

Dosages of the substance of the present invention can vary between wide limits, depending upon the disease or disorder to be treated, the severity of the condition, and the age and health of the individual to be treated, etc. and a physician will ultimately determine appropriate dosages to be used.

This dosage may be repeated as often as appropriate. If side effects develop the amount and/or frequency of the dosage can be reduced or otherwise altered or modified, in accordance with normal clinical practice.

Such compositions may be formulated for human or for veterinary medicine. The present application should be interpreted as applying equally to humans as well as to animals, unless the context clearly implies otherwise.

According to a second aspect of the invention there is provided a method for the treatment of a condition characterised by skin ischaemia, comprising the administration of a composition comprising a pharmacologically acceptable acidifying agent, a pharmacologically acceptable source of nitrite ions or a nitrite precursor therefore.

According to a third aspect of the invention there is provided a composition comprising a pharmacologically acceptable acidifying agent, a pharmacologically acceptable source of nitrite ions or a nitrite precursor therefore as a combined preparation for simultaneous, separate or sequential use in the treatment of skin ischaemia.

According to a fourth aspect of the invention there is provided a kit comprising a pharmacologically acceptable acidifying agent and a pharmacologically acceptable source of nitrite ions or a nitrite precursor therefore for use as a combined preparation in the treatment of skin ischaemia.

According to a fifth aspect of the present invention there is provided a membrane comprising a pharmacologically acceptable acidifying agent and a pharmacologically acceptable source of nitrite ions or a nitrite precursor therefore. The membrane may be fully-, or partially-permeable, including semi-permeable or selectively permeable, to the passage of nitric oxide. Such membranes can prevent direct contact of the composition with the skin but can permit diffusion of nitric oxides into the skin.

This is particularly advantageous in the treatment of areas of broken skin, open wounds or serious bums. In this way the integrity of the wound area is preserved. Suitable membranes include, but are not limited to, polymeric materials such as nitrocellulose, cellulose, agarose, alginate gels, polyethylene, polyester (e.g. a hydrophilic polyester block copolymer) etc. A suitable membrane that can be used in practice is Sympatex™ which is composed of fibers of hydrophilic polyester block copolymer. The present invention therefore extends to the use of such membranes in the treatment of these and other disease conditions, for example skin ischaemia and/or microbial infections, e.g. bacterial, yeast or fungal infections.

Preferred features for the second and subsequent aspects of the invention are as for the first aspect mutatis mutandis.

### BRIEF DESCRIPTION OF THE DRAWINGS

FIG. 1 shows the effect of direct application and subsequent removal of the treatment on the microcirculatory blood flow in forearm skin and finger pulps of healthy subjects in accordance with one embodiment of the invention.

FIG. 2 shows the effect of direct application and subsequent removal of the treatment on the microcirculatory blood flow in forearm skin and finger pulps of subjects with severe Raynaud's phenomenon.

FIG. 3 shows nitric oxide diffusion through a selection of membranes where the vertical axis shows nitric oxide concentration and the horizontal axis in the time in minutes. FIG. 3a shows the results using Saranwrap™ (SW-01) and FIG. 3b shows the results using clingfilm (CF-02).

FIG. 4 shows the diffusion effect of the treatment through a membrane on the forearm skin microcirculatory blood flow in a healthy subject.

FIG. 5 shows the diffusion effect of the treatment through a membrane on forearm skin microcirculatory blood flow in a healthy subject.

FIGS. 6(a)-(i) show the transmembrane diffusion for sodium nitrite and ascorbic acid in 0.8% agar gel, using 1% sodium chloride as an intermediate at final concentrations of 500 mM, 250 mM, 165 mM, 50 mM, 25 mM, 5 mM, 2.5 mM and 0.5 mM, respectively.

FIG. 7 shows the results of the application of nitric oxide generating gel.

FIG. 8 shows the anti-microbial properties of the NO-generation gel at different nitrite ion concentrations against Staphylococcus aureus NCTC9353 and Escherichia coli NCTC10148.

### DETAILED DESCRIPTION

The invention will now be described, by way of illustration only with reference to the following examples and figures which are provided for the purposes of illustration and are not to be construed as being limiting on the invention.

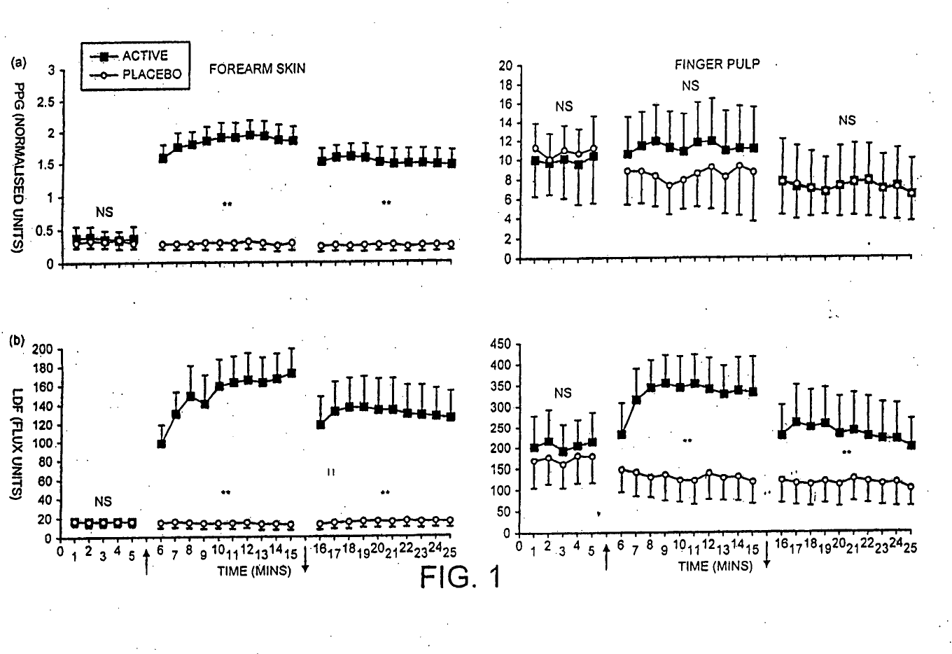

FIG. 1 shows the effect of direct application and subsequent removal of the treatment on the microcirculatory blood flow in forearm skin and finger pulps of healthy subjects. The vertical axes are blood flow, photoplethysmography (PPG) relating to microcirculatory volume and laser Doppler fluximetry (LDF) which relates relating to microcirculatory flux (red blood cell count.times.velocity). The horizontal axis is the time in minutes; NS=not significant; points shown represent the mean value; error bars are 95% confidence;*=p<0.05;**=p<0.01;***=p<0.001; up arrow=application of gel; and down arrow=removal of gel.

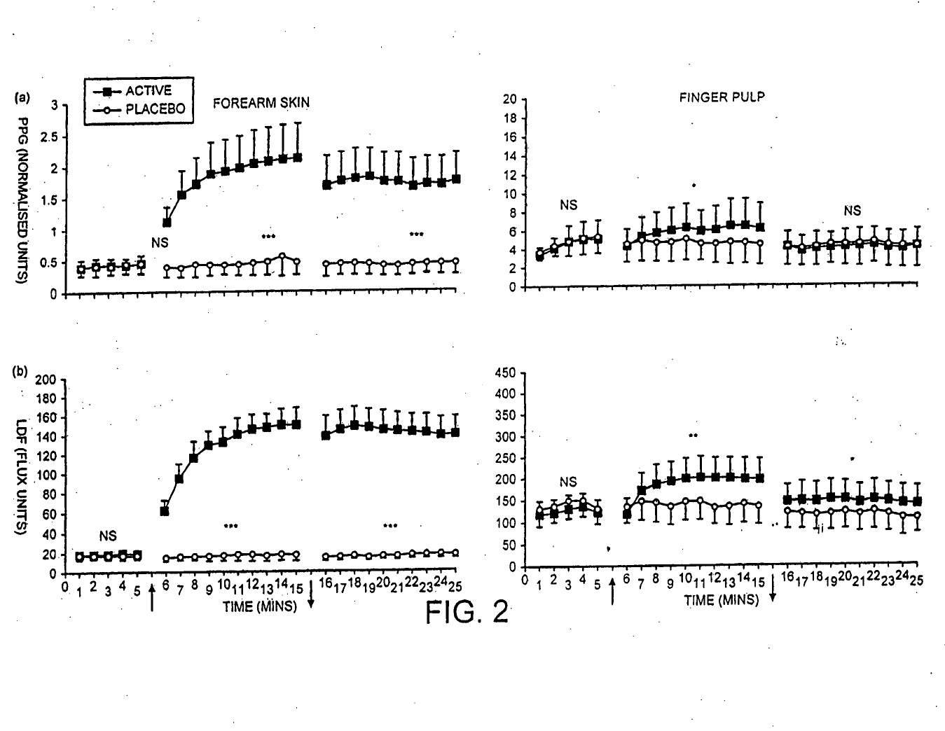

FIG. 2 shows the effect of direct application and subsequent removal of the treatment on the microcirculatory blood flow in forearm skin and finger pulps of subjects with severe Raynaud's phenomenon. The vertical axes are blood flow, photoplethysmography (PPG) relating to microcirculatory volume and laser Doppler fluximetry (LDF) which relates to microcirculatory flux. The horizontal axis is the time in minutes.

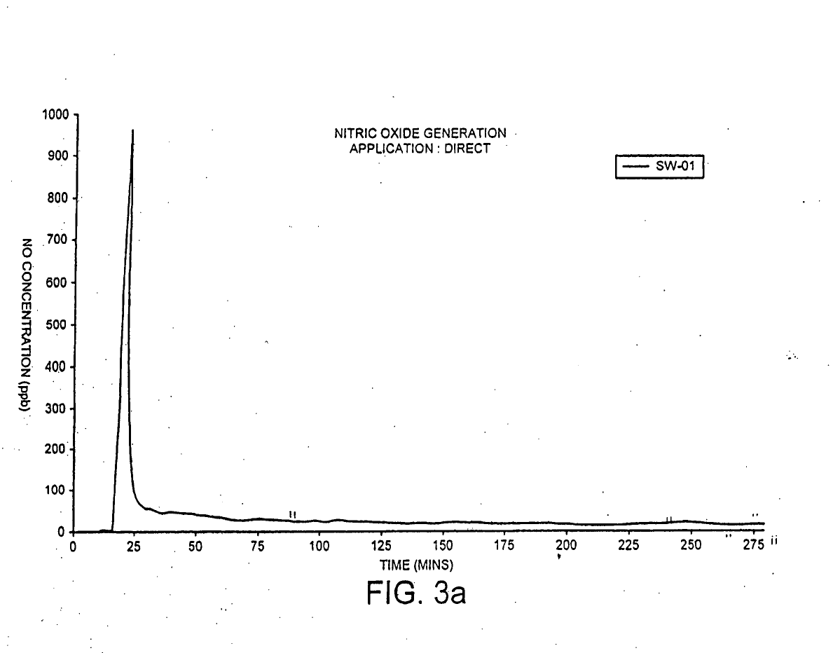

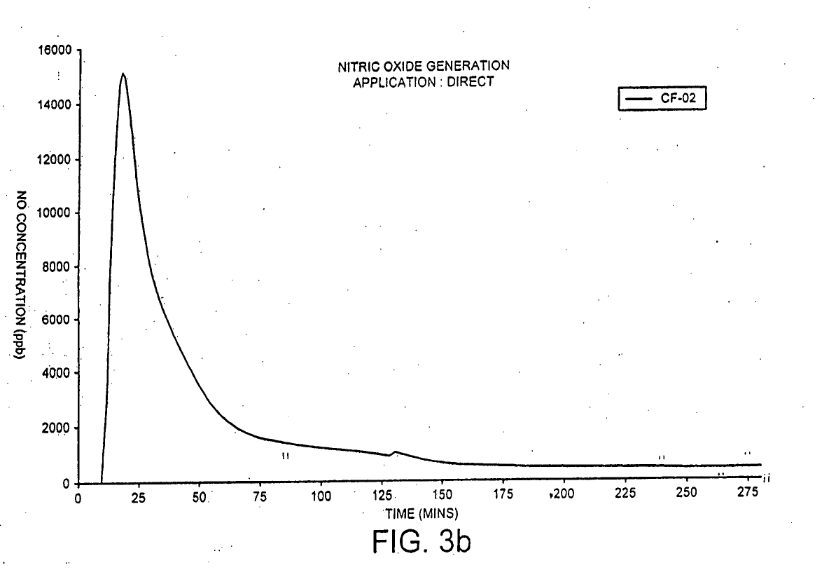

FIG. 3 shows nitric oxide diffusion through a selection of membranes where the vertical axis shows nitric oxide concentration and the horizontal axis in the time in minutes. FIG. 3a shows the results using Saranwrap™ (SW-01) and FIG. 3b shows the results using clingfilm (CF-02).

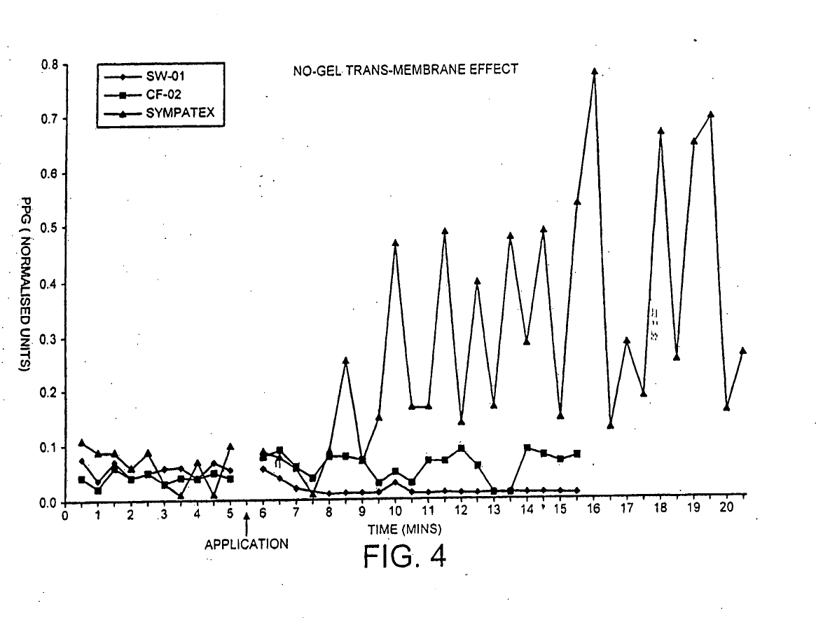

FIG. 4 shows the diffusion effect of the treatment through a membrane on the forearm skin microcirculatory blood flow in a healthy subject. The vertical axis is blood flow, photoplethysmography (PPG) relating to microcirculatory volume and the horizontal axis is the time in minutes.

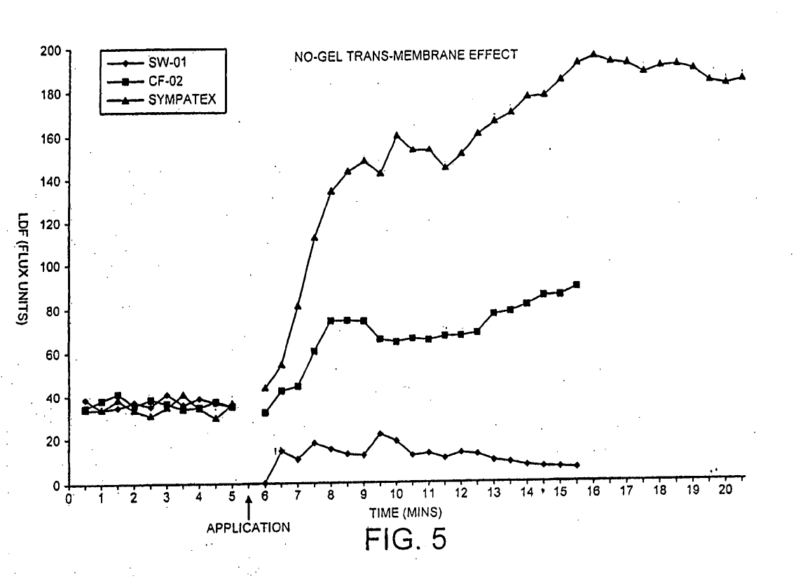

FIG. 5 shows the diffusion effect of the treatment through a membrane on forearm skin microcirculatory blood flow in a healthy subject. The vertical axis is blood flow, laser Doppler fluximetry (LDF) relating to microcirculatory flux and the horizontal axis is the time in minutes.

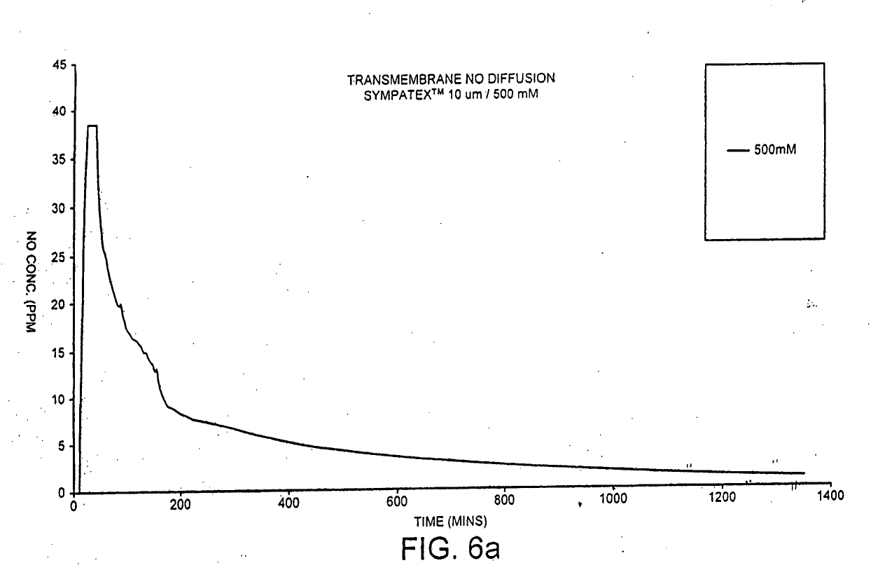

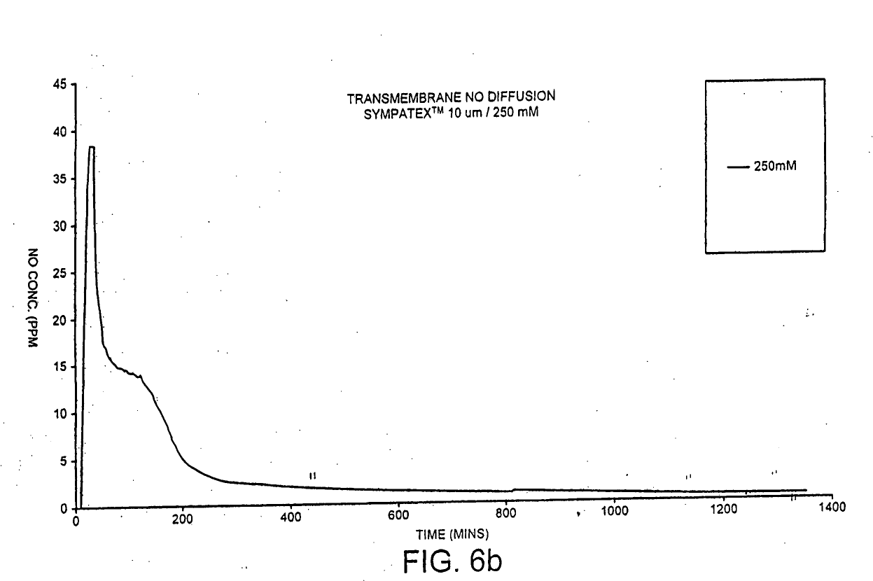

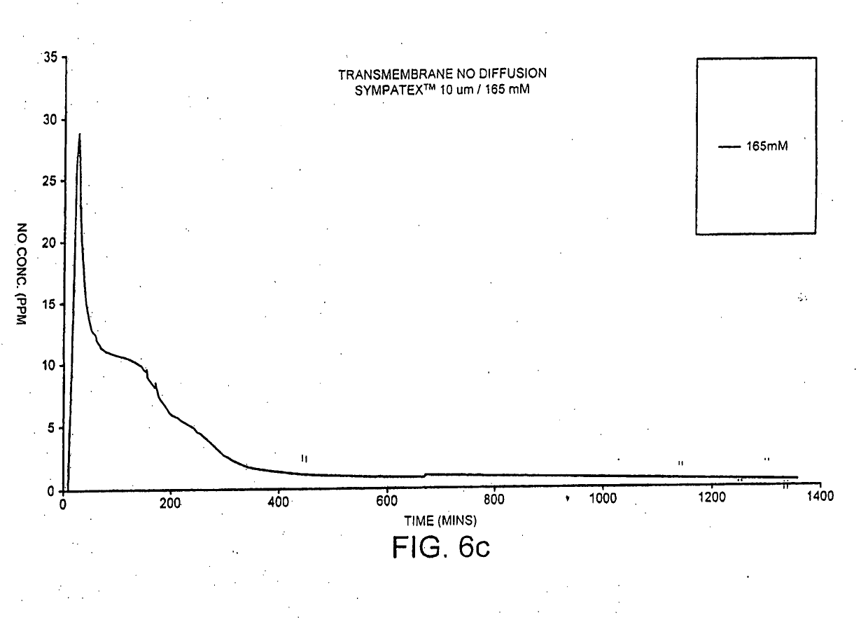

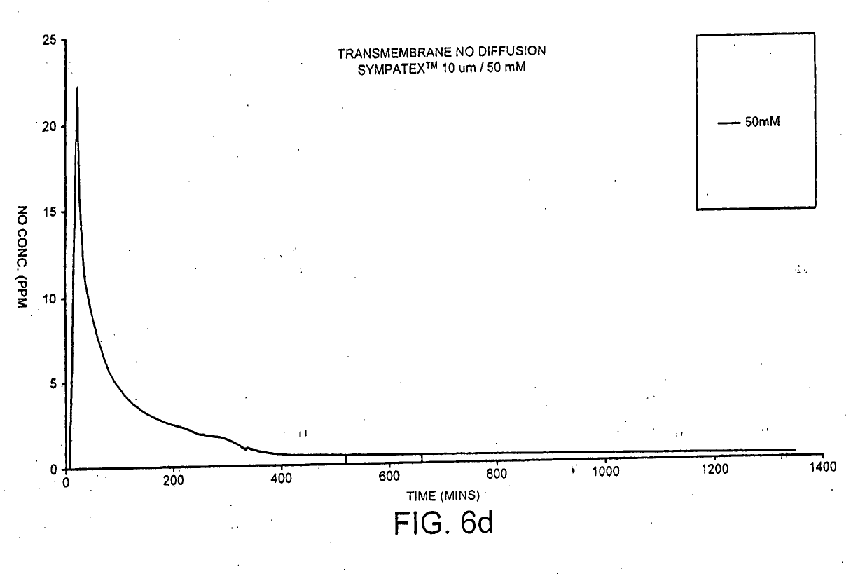

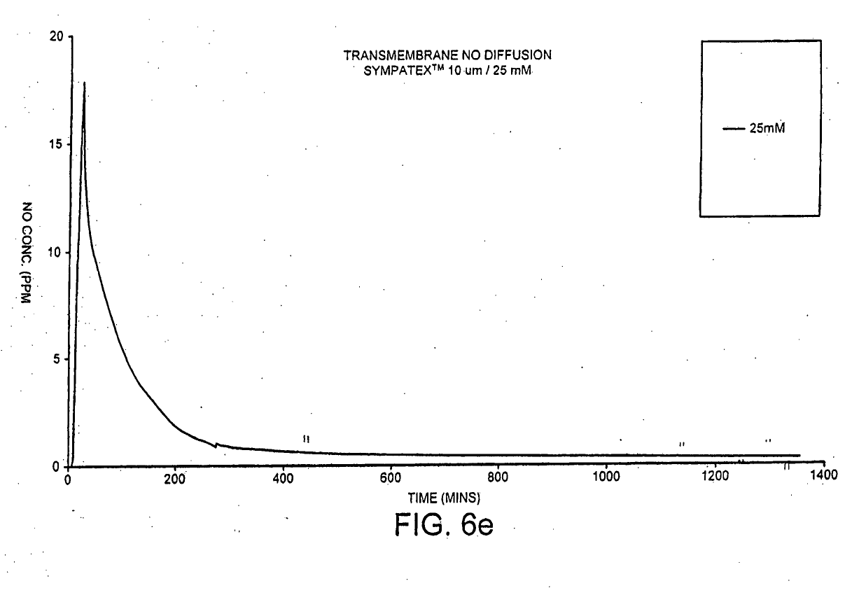

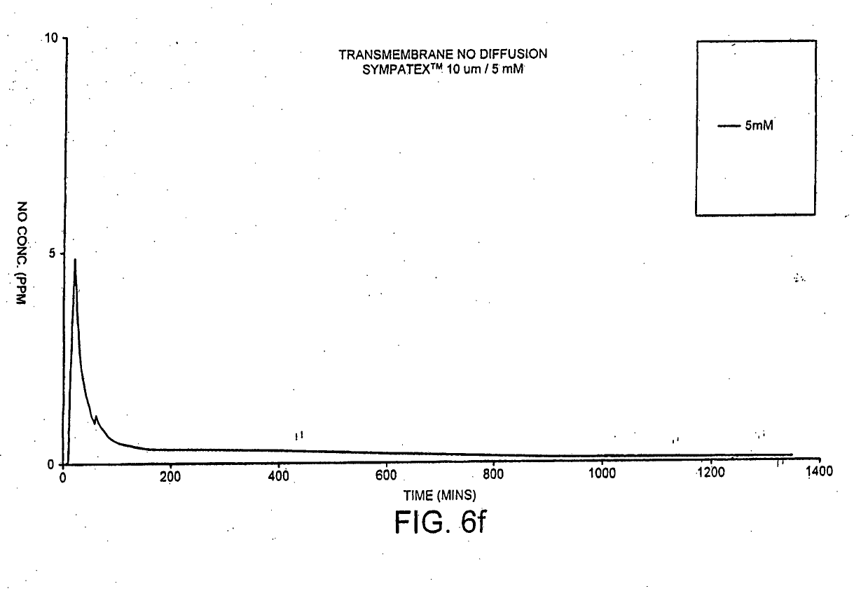

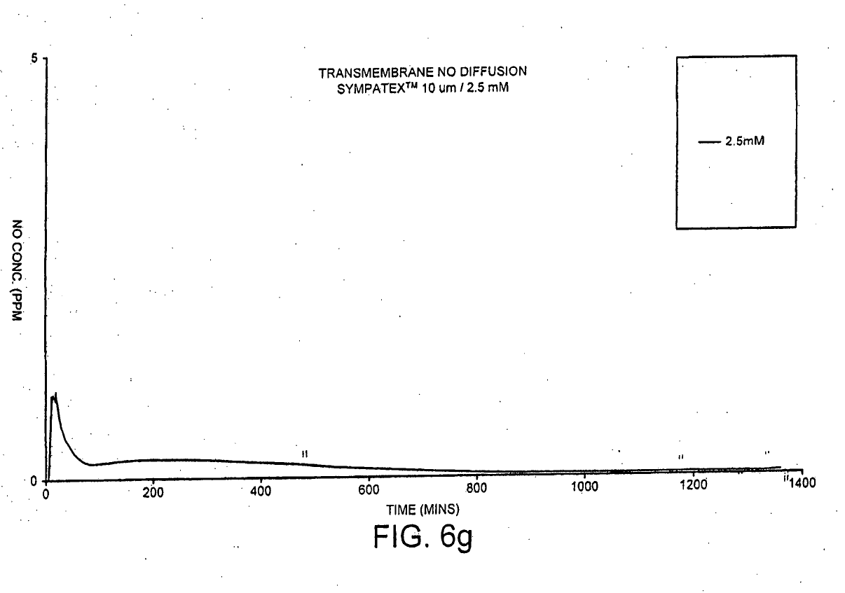

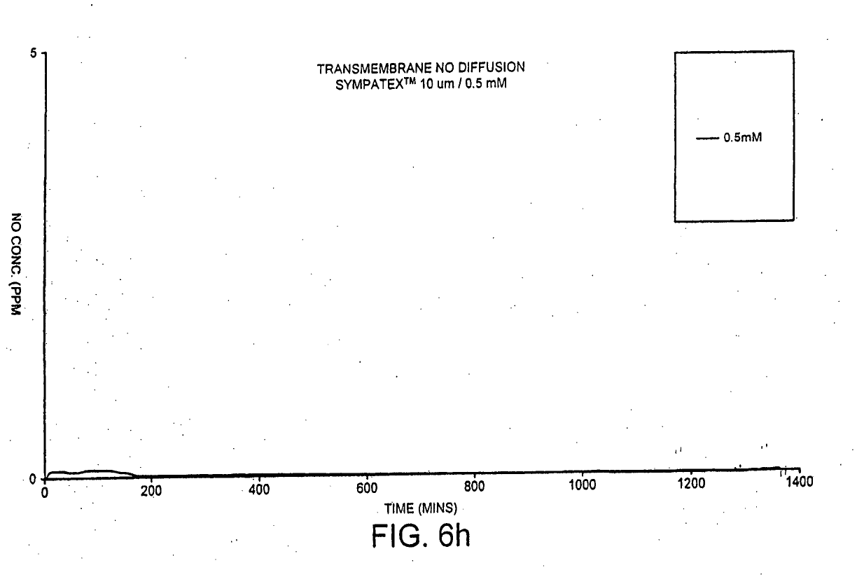

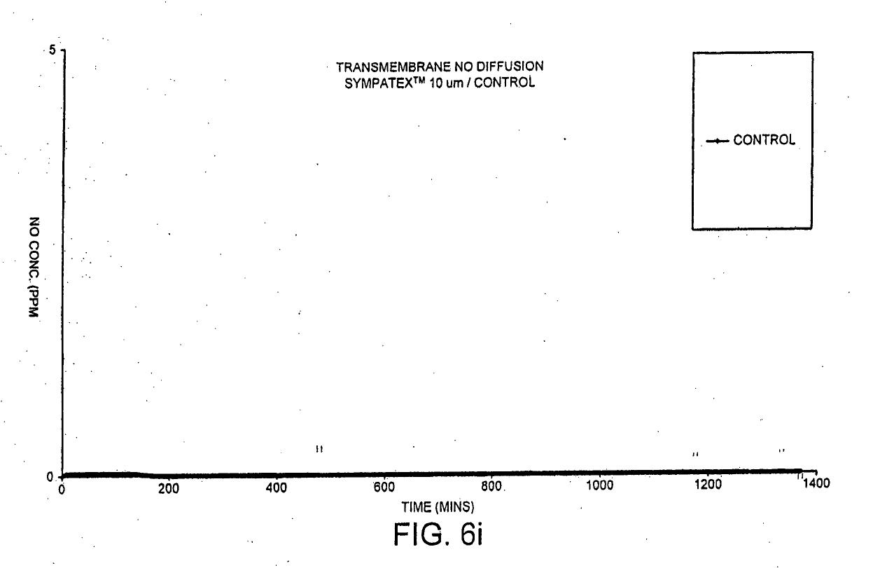

FIGS. 6(a)-(i) show the transmembrane diffusion for sodium nitrite and ascorbic acid in 0.8% agar gel, using 1% sodium chloride as an intermediate at final concentrations of 500 mM, 250 mM, 165 mM, 50 mM, 25 mM, 5 mM, 2.5 mM and 0.5 mM. A control of nitrite and 0.8% agar gel using 1% sodium chloride as an intermediate was also used. The figure illustrates nitric oxide diffusion through Sympatex™ 10 μm (Akzo Nobel) membrane where the vertical axis shows the nitric oxide concentration in parts per million (PPM) and the horizontal axis shows the time in minutes. In FIGS. 6(a) and 6(b) the initial peaks are artificially flattened due to the full scale deflection of the detection device.

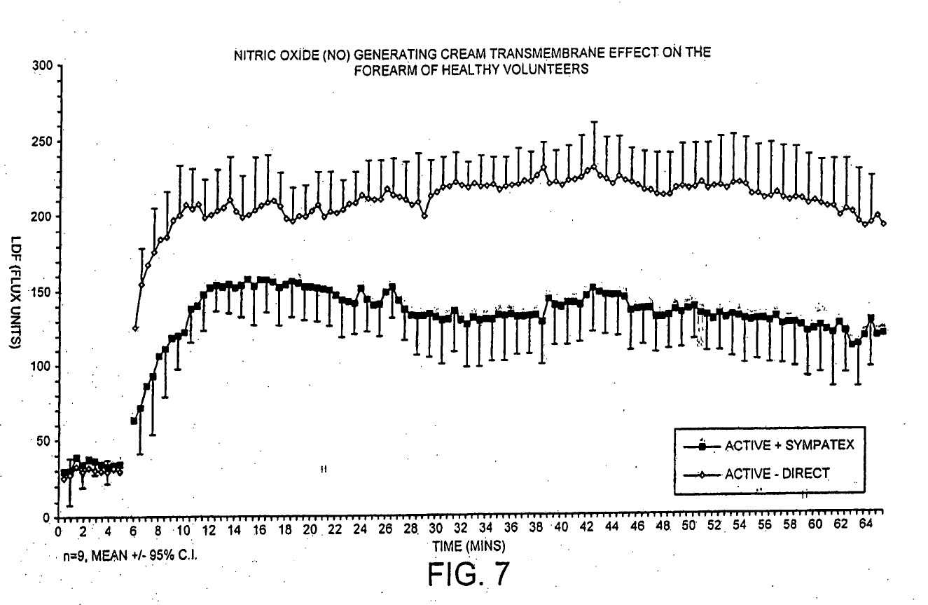

FIG. 7 shows the results of the application of nitric oxide generating gel consisting of 330 mM of sodium nitrite and ascorbic acid in KY jelly™ to the forearm skin and simultaneously to Sympatex™ 10 μm membrane (Akzo Nobel), which was then applied to the forearm skin of the contralateral limb if nine healthy subjects. Conditions and experimental methods were the same as used for the application of the NO-generation gel on healthy subjects in FIGS. 1, 2, 4 and 5. The vertical axis shows Laser Doppler Fluximetry units and the horizontal axis shows the time in minutes.

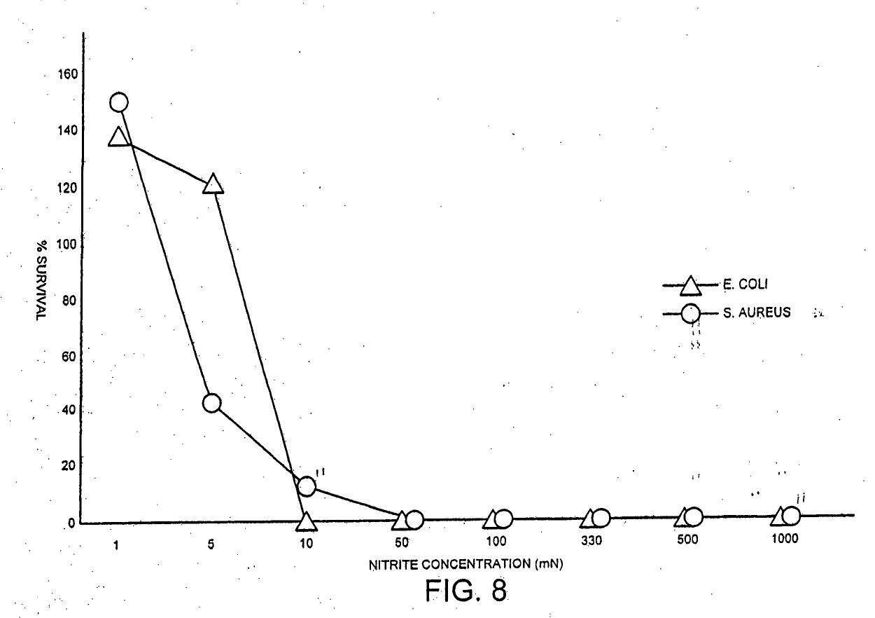

FIG. 8 shows the anti-microbial properties of the NO-generation gel at different nitrite ion concentrations against Staphylococcus aureus NCTC9353 and Escherichia coli NCTC10148. The vertical axis shows microbial survival as a percentage and the horizontal axis shows NO-gel concentration in mM.

### EXAMPLE 1

Microcirculatory Response to Topical Application of NO-Generating Gel in Healthy Subjects

A nitric oxide-generating gel (NO-generating gel) was prepared as follows. Sodium nitrite (Analar™ grade from Sigma, Poole, Dorset, UK) was added to KY Jelly™ (Johnson & Johnson) to make a 5% w/w solution. Ascorbic acid (Sigma) was also added to KY Jelly™ (Johnson & Johnson) to make a 5% w/w solution. Approximately 0.5 ml of each solution was mixed together on the skin of a patient using a sterile swab. When the two solutions are brought into contact, the ensuing reaction leads to the generation of nitric oxide. The reaction may be stopped by cleaning the skin with paper or a swab soaked in ethyl alcohol.

With reference to FIG. 1 the microcirculatory response to topical application of NO-generating gel was measured in 10 healthy subjects. The effect of placebo treatment was measured simultaneously on the contra-lateral limb. The skin microcirculatory volume was measured by infra-red photoplethysmography [PPG] and microcirculatory velocity by laser Doppler fluximetry [LDF]. All examinations were performed in a quiet, draught-free, temperature and humidity controlled laboratory (24° C.±1°. relative humidity 30-40%) in the morning at approximately the same time of day for each subject.

Placebo treatment did not have any effect upon microcirculatory blood flow in either the forearm or the finger of the normal subjects. The vasodilator response to the active treatment reached a plateau phase in all patients within the ten minutes of active gel application. Forearm skin and finger pulp blood flow increased markedly following topical application of a NO-generating gel in the healthy volunteers. When the active gel was applied to the forearm skin all subjects showed a large vasodilator response to active gel treatment in both volume and flux. This increase in blood flow was sustained after removal of the active gel. The active gel had no significant effect on finger microcirculatory volume (PPG) (FIG. 1: Finger pulp), however microcirculatory flux increased significantly (p<0.01) and remained so after removal (p<0.01; FIG. 1: Finger pulp).

### EXAMPLE 2

Microcirculatory Response to Topical Application of NO-Generating Gel in Patients with Severe Primary Vasospasm

FIG. 2 shows the microcirculatory response to topical application of NO-generating gel was measured in 20 patients with severe primary vasospasm. The effect of the placebo treatment was measured simultaneously on the contra-lateral limb. Conditions were the same as those used for the application of the treatment on healthy subjects in FIG. 1. The skin microcirculatory volume was measured by infra-red photoplethysmography [PPG] and microcirculatory velocity by laser Doppler fluximetry [LDF].

Placebo treatment did not have any effect upon microcirculatory blood flow in either the forearm or the finger of any patients. The vasodilator response to the active treatment reached a plateau phase in all patients within ten minutes of the application of active gel. When the gel was applied to the forearm skin all patients showed a large vasodilator response to active gel treatment in both volume and flux. This increase in blood flow was sustained after removal of the active gel in both groups (FIG. 2: Forearm and finger pulp). The active gel to the finger pulp caused a significant increase in microcirculatory volume p<0.05), which returned rapidly to the resting level on removal of the gel. Active gel also significantly increased finger microcirculatory flux (p<0.01) which achieved normal values. This increase was sustained, although reduced, after removal of the gel (p<0.05).

### EXAMPLE 3

Generation of Nitric Oxide Derived through a Membrane

FIG. 3 shows the generation of nitric oxide derived from the reaction previously detailed through a membrane. Nitric oxide concentrations were measured by a nitric oxide sensitive meter: Model 42C Chemiluminescence NO—NO₂—NOₓ analyser Thermo Environmental Instruments Inc., Mass. USA) connected to a data acquisition system and IBM computer. Measurements were made continually and readings were taken every 10 seconds for 275 minutes. Material 1 was domestic clingfilm, Material 2 was Saranwrap™ (Sigma) and Material 3 was (Sympatex™, Akzo Nobel).

### EXAMPLE 4

Microcirculatory Response of the Application of NO-Generating Gel to Three Differing Membrane Materials

FIG. 4 shows the microcirculatory response of the application of NO-generating gel to three differing membranes which were then applied to the forearm skin of a healthy subject. Conditions were the same as those used for the application of the treatment upon healthy subjects in FIG. 1. The skin microcirculatory volume was measured by infrared photoplethysmography [PPG]. Material 1 was domestic clingfilm, Material 2 was SaranwrapTM (Sigma) and Material 3 was (Syrnpatex™, Akzo Nobel).

The increase in microcirculatory blood volume is a reflection of the diffusion of nitric oxide through the membrane towards the skin. The transfer of nitric oxide through the membrane is a reflection of the physical characteristics of the material and is highly variable. Material number 3 (Sympatex™, Akzo Nobel) had a superior diffusion profile.

### EXAMPLE 5

Microcirculatory Response of the Application of NO-Generating Gel to Three Differing Membrane Materials

FIG. 5 shows the microcirculatory response of the application of NO-generating gel to three differing membranes which were then applied to the forearm skin of a healthy subject. Conditions were the same as those used for the application of the treatment on healthy subjects in FIG. 1. The skin microcirculatory velocity was measured by laser Doppler fluximetry [LDF].

The increase in microcirculatory velocity is a reflection of the diffusion of nitric oxide through the membrane towards the skin. The transfer the nitric oxide through the membrane is a reflection of the physical characteristics of the material and is highly variable. Material number 3 (Sympatex™, Akzo Nobel) had a superior diffusion profile.

### EXAMPLE 6

Comparison of Nitric Oxide Generation through a Membrane

FIG. 6 shows the generation of nitric oxide derived from the reaction described above through a 10 μm Sympatex™ membrane. Nitric oxide concentrations were measured by a nitric oxide sensitive meter: Model 42C chemiluminescence NO—NO₂—NOₓ analyser (Thermo Environmental Instrumental Inc., Mass., USA) connected to a data acquisition system and an IBM computer. Measurements were made continually and readings were taken every 10 seconds for 1350 minutes.

The results shown in FIG. 6 illustrate that the transmembrane diffusion coefficient is closely related to the production of nitric oxide, which is a direct product of the concentration of both the source of the nitrite ions and the acidifying agent.

Furthermore, the results demonstrate that a basal production of nitric oxide is sustained for a significant period of time after mixing the reagents.

### EXAMPLE 7

Microcirculatory Response of the Application of NO-Generating Gel

The nitric oxide generating gel consisting of 330 mM of both sodium nitrite and ascorbic acid in KY jelly™ was applied directly to the forearm skin and simultaneously to Sympatex™ 10 μm membrane (Akzo Nobel), which was then applied to the forearm skin of the contralateral limb if nine healthy subjects. Conditions and experimental methods were the same as used for the application of the NO-generation gel on healthy subjects in FIGS. 1, 2, 4 and 5. The results are shown in FIG. 7. It should be noted that in FIG. 7 that the concentrations of the admixture are in a different unit form (i.e., mM instead of % w/w). Laser Doppler Fluximetry (LDF) measured the skin microcirculatory. flux.

The statistically significant increase in microcirculatory flux from baseline was a reflection of the diffusion of nitric oxide through the membrane towards the skin. This vasodilation, indicated by LDF through the membrane ranged from 60-75% (mean 64%) of that observed when the NO-generation gel was applied directly to the skin of the forearm. The results shown in FIG. 7 support the observations described in FIG. 1 which show that the vasodilator response to the direct treatment reached a plateau phase in all patients within 10 minutes of gel application. A plateau phase, although reduced in amplitude was achieved within 16 minutes when the NO-generation gel was applied to the membrane and reflects a lag phase which is related to membrane diffusion characteristics.

### EXAMPLE 8

Anti-Microbial Properties of NO-Generation Gel

The antimicrobial properties of NO-generation gel after diffusion through a 10 μm Sympatex™ membrane were investigated as follows. NO was generated by an admixture of sodium nitrite and ascorbic acid in 0.8% agar gel, using 1% sodium chloride as an intermediate. The preparation was tested on S. aureus NCTC9353 and E. coli NCTC10148 using a range of concentrations of sodium nitrite and ascorbic acid. Cultures of S. aureus and E. coli were prepared by innoculating 20 ml of LB (Luria-Bertani 10 g Bacto-Tryptone, 5 g Bacto-Yeast extract and 10 g/l sodium chloride at pH7.5) broth with 2-3 colonies, and incubated at 37° C. overnight. 24 ml of 1.5% agar in NaCl were innoculated with 1 ml of either S. aureus or E. coli and poured into Petri dishes. Discs of membrane (100 mm in diameter) were sterilised in 70% ethanol and the discs were then placed in a lamina flow cabinet to allow the ethanol to evaporate. 5 ml of 0.8% agar in 1% NaCl, containing either sodium nitrite or ascorbic acid at final concentrations of 500 mM, 250 mM, 165 mM, 50 mM, 25 mM, 5 mM, 2.5 mM and 0.5 mM were prepared. Final concentrations in use are halved.

In the center of sterile inverted Petri dish lids, 1 ml of each concentration of sodium nitrite and ascorbic acid was added and mixed. Disinfected membrane was then placed over the top of this immediately, using sterilised forceps. The membrane was carefully positioned so that it hung over the edge of the lid equally in all directions. The base of the Petri dish was then placed upside down on top of the lid/mixture/membrane arrangement ensuring that a 2-3 mm gap was left between the membrane and the inverted innoculated agar.

The apparatus was incubated overnight at 36° C. after which it was removed. The base of the Petri dish (upside down) was removed and the central area of agar sampled by cutting a circle using a sterile plastic measuring cup. The agar was then macerated in 10 ml of LB broth and 5 ml of sterile glass beads. Serial dilutions were carried out and the samples plated onto blood agar plates that were incubated for 24 hours at 36° C. The surviving colonies were then counted.

Anti-microbial properties of nitric oxide were seen at concentrations of nitrite above 50 mM. Below this concentration partial or no anti-microbial activity was seen. Above this concentration, cell lysis was complete resulting in complete killing of the bacteria. The results shown in FIG. 8 illustrate the anti-microbial effect of varying concentrations of NO-generation gel and resulting diffusion through Sympatex™ 10 μm membrane.

1. A method for treating skin ischaemia, comprising:
locating the skin ischaemia; and
administering a composition consisting essentially of a pharmacologically acceptable acidifying agent and a pharmacologically acceptable source of nitrite, wherein the pharmacologically acceptable acidifying agent is selected to reduce a pH value of the composition to 4 or below.

2. The method as claimed in claim 1, wherein administering the composition comprises applying the composition to a gas permeable membrane disposed on a patient.

3. The method of claim 1, wherein the skin ischaemia comprises one selected from the group consisting of skin ulcers and post-operative trauma.

4. The method of claim 1, wherein the pharmacologically acceptable acidifying agent is selected to reduce a pH value at a site of application to between 2 and 4.

5. The method of claim 1, wherein the pharmacologically acceptable acidifying agent is selected to reduce a pH value at a site of application to around 4.

6. A method for treating skin ischaemia, comprising:
providing a composition consisting essentially of a pharmacologically acceptable acidifying agent and a pharmacologically acceptable source of nitrite, wherein the pharmacologically acceptable acidifying agent is selected to reduce a pH value of the composition to 4 or below; and
administering the composition as a combined preparation to treat the skin ischaemia in a manner selected from the group consisting of simultaneous, separate, and sequential use.

7. The method of claim 6, wherein the skin ischaemia comprises one selected from the group consisting of skin ulcers and post-operative trauma.

8. The method of claim 6, wherein the pharmacologically acceptable acidifying agent is selected to reduce a pH value at a site of application to between 2 and 4.

9. The method of claim 6, wherein the pharmacologically acceptable acidifying agent is selected to reduce a pH value at a site of application to around 4.
